(19) United States
(12) Patent Application Publication
(10) Pub. No.: US 2009/0025732 A1
(43) Pub. Date: Jan. 29, 2009

(54) Method and device for generating a passive movement in a diagnostic device

(76) Inventor: Ilan Elias, Frankfurt am main (DE)

Correspondence Address: CANTOR COLBURN, LLP, 20 Church Street, 22nd Floor, Hartford, CT, 06103, US

(21) Appl. No.: 12/220,191

(22) Filed: Jul. 22, 2008

Related U.S. Application Data
(62) Division of application No. 10/375,717, filed on Feb. 27, 2003.

(30) Foreign Application Priority Data
Apr. 1, 2002 (DE) 102 14 798.1
Apr. 1, 2002 (DE) 202 05 012.2
Aug. 6, 2002 (DE) 102 35 963.6

(51) Int. Cl. A61B 5/103; A61G 15/00

(52) U.S. Cl. 128/845; 600/587

(57) ABSTRACT

A method and an apparatus for generating a passive movement of a patient in a magnetic resonance tomograph. The apparatus includes a support for supporting at least one body part of the patient. The support, together with a patient bed can be moved into a passage of the magnetic resonance tomograph. Driven by a motor, the support can be swiveled about at least one axis inside the passage of the magnetic resonance tomograph. The components of the support and its drive, which are disposed inside the passage of the magnetic resonance tomograph, are made of non-ferromagnetic materials.

### CROSS-REFERENCE TO RELATED APPLICATIONS

This application is a divisional application of prior application Ser. No. 10/375,717 filed Feb. 27, 2003, the entire contents of which are hereby incorporated by reference. This application claims the benefit of a priority under 35 USC 119 (a)-(d) to German Patent Application No. 102 14 798.1 filed Apr. 1, 2002 and German Patent Application No. 202 05 012.2 filed Apr. 1, 2002 and German Patent Application No. 102 35 963.6 filed Aug. 6, 2002, the entire contents of each which are hereby incorporated by reference.

### BACKGROUND OF THE INVENTION

1. Field of the Invention

The present application relates to a method and apparatus for generating a passive movement of a patient in a magnetic resonance tomograph with a support for supporting at least one body part of the patient, which support together with a patient bed or the like can be moved into an imaging passage of the magnetic resonance tomograph.

2. The Prior Art

To provide for an optimum diagnosis of injuries and disorders of joint, such as an ankle joint it is known to scan a patient of at least relevant body parts by means of a magnetic resonance tomograph (MRT) and to make a diagnosis on the basis of the images obtained. For this purpose, it is frequently required to position or to be able to move the body part concerned in different and defined ways as required. Therefore, apart from static instantaneous images, static images have so far been produced in various joint positions. The joint positions are adjusted manually via a mechanism, and viewing the resulting images is effected by means of an image loop (cine-mode). However, this is particularly time-consuming and therefore can only be applied in individual cases. Thus the possibilities of a modern magnetic resonance tomograph, which also provides for very fast image recordings, are not utilized. Hence, the diagnostics frequently indispensable with a magnetic resonance tomograph, which can excellently represent both the osseous and the cartilaginous parts of the human body as well as its soft-tissue structures, is not being used optimally.

In addition, there is the disadvantage that despite this presently most modern form of diagnosis not all disorders or injuries and damage to the human body can be detected by static instantaneous images, in particular as it has not been possible so far to make images in movement of a sufficient quality. However, this impairs the clinical diagnostics, as thus the probability (sensitivity) to diagnose existing pathological findings with diverse structures is not yet optimally possible or partly not possible. In magnetic resonance tomograph, in contrast to X-ray diagnostics, there have so far not been any firmly predefined and reproducible settings to provide for the representation of real-time movements.

This in particular due to the fact that as a result of the strong magnetic field in a magnetic resonance tomography, the use of conventional movement apparatuses is not possible. Conventional electromechanical motors, which are usually employed in movement apparatuses, consist of magnets and coils which in the strong magnetic fields of (0.2-3 Tesla) of a magnetic resonance tomograph deflect particularly strongly and thus lead to image distortions, so-called image artefacts. Once such image artefacts occur, however, a proper diagnosis no longer is possible.

### BRIEF DESCRIPTION OF THE INVENTION

Therefore, an embodiment of the invention provides a method and an apparatus for a defined reproducible passive movement of a patient in a magnetic resonance tomography without producing image distortions, i.e., image artifacts, which make a diagnosis impossible.

An embodiment of the invention, provides that driven by a motor, the support can be swiveled about at least one axis inside the imaging passage of the magnetic resonance tomograph, and that the components of the support and its drive, which are disposed inside the imaging passage of the magnetic resonance tomograph, are made of non-ferromagnetic materials. Due to this structure, the sensitivity of the magnetic resonance diagnostics with respect to the examination of the joints is increased distinctly. Thus predefined continuous movements can be set and the movements can be reproduced, in order to be able to represent real-time images (online images). The method and apparatus allows a body part to move in the magnetic resonance tomograph within the full scope of movement, passively and as desired for the respective diagnostic situation. Since the diagnostic possibilities have thus been improved and expanded, suspected injuries and the like can be detected and verified far better than has so far been possible. Since the components used in the imaging passage of the magnetic resonance tomography are formed of non-ferromagnetic materials, the occurrence of image artefacts, which make a diagnosis more difficult, is reduced in addition.

In an embodiment the occurrence of image artifacts can furthermore be prevented in that the drive for swiveling the support is effected by means of a piezoelectric motor.

Preferably, the drive for swiveling the support is controlled by a control unit, which is electrically grounded and shielded from magnetic radiation. In the method and apparatus, the movements can therefore be adjusted electronically and automatically controlled from outside the room of the magnetic resonance tomograph in always the same exactly reproducible positions. The position between the body part to be examined and the magnetic resonance tomograph can remain unchanged during the entire diagnostic procedure. At the same time, however, it is for the first time possible to perform precisely defined movements controlled by motors during the imaging procedure. On the one hand, this allows the magnetic resonance tomograph operator to specifically take a certain image and on the other hand to represent the movement itself in the sense of a real-time image. Beside the use of a piezoelectric motor for driving the support, it is also possible to use pneumatic or hydraulic drives for this purpose.

When the control unit is disposed outside the zone around the magnetic resonance tomograph, in which the operation of the magnetic flux density is ≧0.2 Tesla, the function of the control unit is not impaired by the strong magnetic field of the magnetic resonance tomography. At the same time, image artifacts caused by the control unit can be avoided.

The control unit of the support can further be improved in that the control unit is provided with at least one sensor, in particular with an optical encoder, for detecting the position of the support or of the motors.

To be able to avoid the occurrence of image artefacts, which make diagnosis more difficult, even more effectively, the control unit can be connected with the drive of the support and possibly with the sensors via electrically grounded and shielded lines, which outside the imaging passage of the magnetic resonance tomograph are provided with ferrites.

In accordance with another embodiment of the invention, the support when driven by a motor, can independently be swiveled about two axes. Thus, the physiological movements of the body parts to be examined can be represented even better.

The physiological movement of an ankle joint can be imitated particularly well with the apparatus when the support can be swiveled about a first horizontal axis and a second axis inclined with respect to the vertical by about 35° in the horizontal plane and by about 18° in the sagittal plane. This inclination of the second axis corresponds to the average geometrical axis of the lower ankle joint as determined by Van den Bogard.

The pressure forces acting on the ankle joint, etc., for instance when running, can be imitated during the examination in the magnetic resonance tomograph in that there are provided means for fixing the at least one body part of the patient on the support, and that at least portions of the support can be moved relative to the fixing means. Preferably, the support can be moved pneumatically or hydraulically relative to the means for fixing the body part. In this way, a stepwise compression of the body part to be examined can occur, which likewise leads to a change in the configuration of the individual parts of the body, which imitate the loads acting for instance when running or the like.

### BRIEF DESCRIPTION OF THE DRAWINGS

Other features of an embodiment of the present invention will become apparent from the following detailed description considered in connection with the accompanying drawings that disclose several embodiments of the invention. It should be understood, however, that the drawings are for the purpose of illustration only and not as a definition of the scope and extant of the invention as disclosed and claimed.

In the following an embodiment of the invention will be described in detail by means of embodiments and with reference to the attached drawings, in which:

FIG. 1 shows a longitudinal section through an imaging passage of a magnetic resonance tomograph with an apparatus arranged on a patient bed;

FIG. 2 shows a sectional view of the apparatus vertical to the sectional plane of FIG. 1; and

FIG. 3 shows a side view of the apparatus shown in FIG. 2.

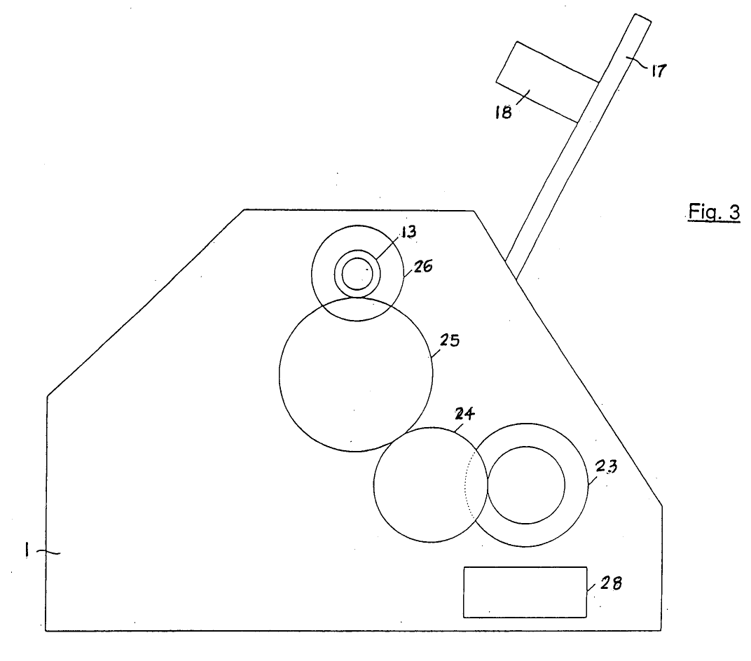

### DETAILED DESCRIPTION OF DISCLOSED EMBODIMENTS

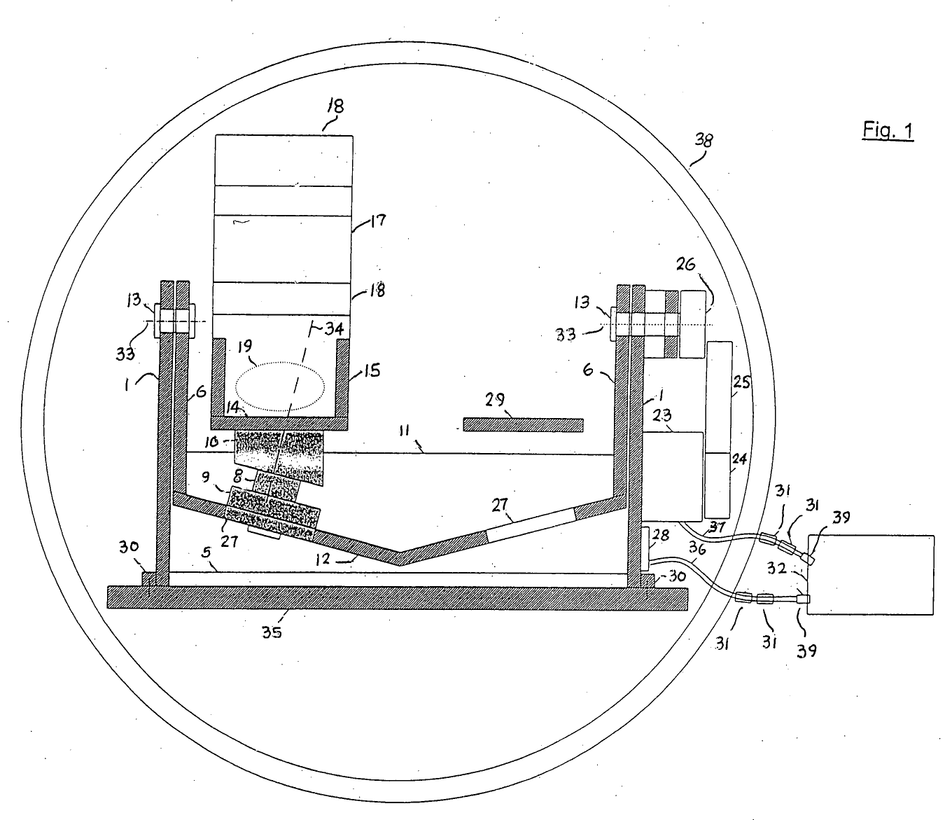

The apparatus represented in the drawings is provided for use in a magnetic resonance tomograph (MRT) for instance with a magnetic flux density between 0.2 and 3.0 Tesla. The magnetic resonance tomograph is indicated in FIG. 1 by an imaging passage 38. In the illustrated embodiment, the apparatus is arranged for examining or imaging the ankle joint. The apparatus has a support 14 for the heel of the foot and a support 17 for the sole of the foot disposed at an angle. The inner and outer malleoli of the foot are stabilized by side walls 15, whereas the support 17 for the sole of the foot is retained on a rear wall 16. In FIG. 1, the foot to be examined is indicated schematically by the reference numeral 19.

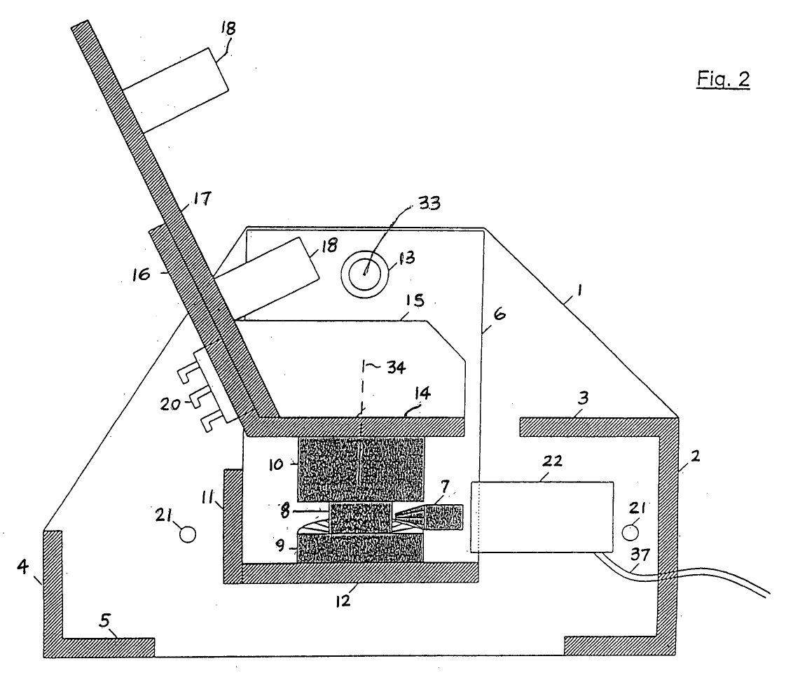

As shown in FIGS. 1 and 2, the apparatus is mounted on a patient bed 35 by means of a connecting unit 30. Together with the patient bed 35, a patient lying on his or her back can be moved into the image passage 38 of the magnetic resonance tomograph, the apparatus being disposed at one end of the patient bed 35. By means of buckles 18, the foot can be maintained fixed in the apparatus. The foot not to be examined can be placed on another support 29 and is not detected in the examination or imaging.

The apparatus disposed on the patient bed 35 furthermore comprises two vertical side walls 1 and a vertical front wall 2 which merges into a horizontal front wall 3. On the side opposite the vertical front wall 2 a vertical rear wall 4 is provided, which is connected with a lower horizontal rear wall 5. Parallel, to the side walls 1 another vertical wall 6 extends, which be means of ball bearings 13 is articulated to the side walls 1 so as to be swiveled about a horizontal axis 33. The vertical walls 6 are connected with each other by a rear wall 11 and a V-shaped bottom wall 12.

In the V-shaped bottom wall 12, two recesses 27 are provided, which can receive a fastening unit 9. Via an intermediate part 8, the fastening unit 9 carries another fastening unit 10 with a gearwheel, on which there is mounted the support 14 for the heel as well as the rear wall 16 with the support 17 of the sole of the foot. The support 14 for the heel as well as the rear wall 16 with the support 17 for the sole of the foot can be rotated relative to the fastening unit 9 via the fastening unit 10. Due to the inclination of the V-shaped bottom wall 12 and the corresponding configuration of the fastening units 9 and 10, the

The materials used for the apparatus are not influenced by the magnetic field of the magnetic resonance tomograph. Therefore they produce no image artefacts that make a diagnosis impossible when non-ferromagnetic materials are used for the components disposed inside the imaging passage 38 of the magnetic resonance tomograph. Such non-ferromagnetic materials include for instance VA4 stainless steel screws and threads, aluminum plates, pins, screws and air-pressure nozzles made of brass, plastic screws, and glass and ceramic ball bearings. The use of semifinished products made of polyoxymethylene (POM) is particularly favorable, as this plastic material absorbs the radiofrequency field (RF) and therefore generates no disturbing radiation.

The illustrated embodiment of the apparatus for generating a passive movement is particularly arranged for the examination or imaging of ankle joints. By means of the motors 22 and 23, the support 14 for the heel as well as the support 17 for the sole of the foot, which is connected with the rear wall 16, can be rotated such that the physiological movement of the ankle joint is imitated. By moving the support 17 for the sole of the foot relative to the rear wall 16 by means of the pressure valve unit 20, a weight load of the foot can be imitated in addition. It is thus possible to make both kinematic and static images from different positions inside the imaging passage 38 of the magnetic resonance tomograph for research and clinical routine diagnosis. As a result, these real-time images of the movements considerably expand the possibilities for using a magnetic resonance tomograph known per se.

Accordingly, while an embodiment of the present invention have been shown and described, it is to be understood that many changes and modifications may be made thereunto without departing from the spirit and scope of the invention as defined in the appended claims.

1-10. (canceled)

11. A device comprising:
at least one first support surface for at least one first body part;
means for stabilizing at least one side of the at least one first body part;
means for fixing the at least one first body part with the at least one first support surface;
means for pivoting the at least one first support surface about a first axis to apply passive movement to the first body part;
means for pivoting the at least one first support surface about a second axis to apply passive movement to the first body part;
means for applying pressure to the at least one first support surface to thereby apply pressure to the at least one first body part; and
means for sensing a pivotal position of the at least one first support surface.

12. The device of claim 11 comprising:
at least one second support surface for a second body part, the means for pivoting about the first axis pivoting the at least one second support surface about the first axis;
the means for pivoting about the second axis pivoting the at least one second support surface about the second axis; and
the means for sensing also sensing the pivotal position of the at least one second support surface.

13. The device of claim 11 wherein the means for pivoting comprises at least one piezoelectric electric motor.

14. The device of claim 11 wherein the means for pivoting comprises at least one pneumatic or hydraulic drive.

15. The device of claim 11 wherein the at least one first support surface can be pivoted independently about the first and second axes.

16. The device of claim 11 wherein the first axis is inclined at about 35 degrees to the second axis.

17. The device of claim 11 wherein the at least one first support surface can be pivoted on the first axis that is inclined at an angle of about 35 degrees to a horizontal plane and inclined at an angle of about 18 degrees in a longitudinal plane relative to a vertical axis.

18. The device of claim 11 wherein the at least one first body part includes a joint of a living or material object.

19. The device of claim 11 wherein the at least one first body part is selected from a group consisting of human, animal or plant.

20. The device of claim 11 wherein the means for sensing detects the position of the means for pivoting.

21. The device of claim 11 wherein the means for sensing detects the pressure applied by the means for applying pressure.

22. The device of claim 11 comprising a non-pivoting support surface for at least one second body part.

23. The device of claim 11 comprising:
opposing first vertical sidewalls;
opposing second vertical sidewalls disposed within the first vertical sidewalls;
the opposing second vertical sidewalls being pivotably connected to the opposing first vertical sidewalls to rotate about the second axis;
one end of each opposing first vertical sidewall being fixed to a platform;
a bottom wall connecting one end of each opposing vertical sidewalls;
at least one recess in the bottom wall; and
a unit positioned in the at least one recess for attaching the at least one first support surface to the bottom wall.

24. The device of claim 23 wherein the attachment unit comprises:
a first attachment unit connected to the at least one first support surface;
a second attachment unit positioned in the at least one recess;
an intermediate part disposed between the first and second attachment units, the intermediate part forming a first gear;
a second gear for engaging with the first gear; and
a first drive motor for driving the second gear to cause the at least one first support surface to pivot about the first axis.

25. The device of claim 23 comprising:
a third gear for pivotably rotating the second vertical sidewalls; and
a second drive motor for driving the third gear to cause the at least one first support surface to pivot about the second axis.

26. The device of claim 22 wherein the material for the at least one first support surface and the at least one second support surface and the means for stabilizing and the means for fixing and the means for pivoting and the means for applying pressure and the means for sensing is chosen to avoid interferences or artifacts in imaging.

27. The device of claim 26 wherein the material is non-ferromagnetic.

28. The device of claim 22 wherein the means for sensing comprises at least one optical encoder.

29. The device of claim 20 wherein the means for sensing comprises at least one optical encoder.

30. The device of claim 21 wherein the means for sensing comprises at least one optical encoder.

31. The device of claim 11 wherein the means for applying pressure is a pneumatic or hydraulic.

32. The device of claims 27 wherein the material is a plastic.

33. The device of claim 11 wherein the means for fixing comprises a detachable buckle.

34. The device of claim 11 wherein the means for applying pressure is continuous or constant or adjustable or step-by-step or variable or a combination thereof.

35. The device of claim 11 wherein the means for stabilizing is opposing sidewalls adjacent respective sides of the at least one first body part.

36. The device of claim 11 comprising:
a control unit disposed in a space separate from the device;
a plurality of electrically grounded and magnetic shielded lines connecting the control unit with the means for pivoting.

37. The device of claim 36 wherein the device is disposed in an imaging modality and the control unit is disposed exterior to the imaging modality.

38. A device comprising:
at least one first support surface for at least one first body part;
opposing sidewall adjacent respective sides of the at least first body part;
the at least one first body part being detachably fixed with the at least one first support surface;
a first drive for pivoting the at least one first support surface about a first axis to apply passive movement to the first body part;
a second drive for pivoting the at least one first support surface about a second axis to apply passive pressure to the first body part;
at least one second support surface for a second body part, the first drive pivoting the at least one second support surface about the first axis;
the second drive pivoting the at least one second support surface about the second axis;
the at least first and second support surfaces being pivoted independently about the first and second axes;
a pneumatic or hydraulic source for applying pressure to the at least one first support surface to thereby apply pressure to the at least one first body part;
means for sensing a pivotal position of the at least one first support surface and the at least one second support surface;
means for sensing the pressure applied by the pneumatic or hydraulic source for applying pressure;
the material for the at least one first support surface and the at least one second support surface and the opposing side walls and the detachable fixing and the first and second drive for pivoting and the source for applying pressure and the means for sensing is chosen to avoid interferences or artifacts in imaging.
a control unit disposed in a space separate from the device;
a plurality of electrically grounded and magnetic shielded lines connecting the control unit with the first and second drives for pivoting.

39. The device of claim 38 wherein the device is disposed in an imaging modality and the control unit is disposed exterior to the imaging modality.

40. A method for using a device for imaging comprising:
providing at least one first support surface for at least one first body part;
stabilizing at least one side of the at least one first body part;
fixing the at least one first body part with the at least one first support surface;
pivoting the at least one first support surface about a first axis to apply passive movement to the first body part;
pivoting the at least one first support surface about a second axis to apply passive movement to the first body part;
applying pressure to the at least one first support surface to thereby apply pressure to the at least one first body part;
sensing a pivotal position of the at least one first support surface.
disposing a control unit in a space separate from the device;
connecting the unit with a plurality of electrically grounded and magnetic shielded lines to control the pivoting;
disposing the device in an imaging modality; and
locating the control unit exterior to the imaging modality.

41. The method of claim 40 wherein the applying pressure is continuous or constant or adjustable or step-by-step or variable or a combination thereof.

42. The method of claim 40 wherein the at least first support surface being pivotable independently about the first and second axes

43. The method of claim 40 comprising the material for the at least one first support surface and for stabilizing and for fixing and for pivoting and for applying pressure and for sensing is chosen to avoid interferences or artifacts in imaging.
